(19) United States
(12) Patent Application Publication
(10) Pub. No.: US 2023/0053497 A1
(43) Pub. Date: Feb. 23, 2023

(54) SYSTEMS AND METHODS FOR PERFORMING EYE-TRACKING

(71) Applicant: META PLATFORMS TECHNOLOGIES, LLC, Menlo Park, CA (US)

(72) Inventors: Syed Shakib Sarwar, Bellevue, WA (US); Barbara De Salvo, Belmont, CA (US); Xinqiao Liu, Medina, WA (US)

(21) Appl. No.: 17/724,302

(22) Filed: Apr. 19, 2022

Related U.S. Application Data
(60) Provisional application No. 63/234,359, filed on Aug. 18, 2021.

(51) Int. Cl. A61B 3/113; G06T 7/246; G06T 7/70

(52) U.S. Cl. CPC A61B 3/113; G06T 7/246; G06T 7/70; G06T 2207/30201

(57) ABSTRACT

The disclosed computer-implemented method may include (i) conditionally operating, at a first frequency, a first stage of an eye-tracking system processing pipeline that detects a region of interest and (ii) operating, at a second frequency that is substantially greater than the first frequency, a second stage of the eye-tracking system processing pipeline that predicts a gaze orientation based at least in part on the detected region of interest. Various other methods, systems, and computer-readable media are also disclosed.

### CROSS REFERENCE TO RELATED APPLICATION

This application claims priority to provisional U.S. Application No. 63/234,359, filed Aug. 18, 2021, the disclosure of which is incorporated, in its entirety, by this reference.

### BRIEF DESCRIPTION OF DRAWINGS

FIG. 1 is an example method for performing eye-tracking.

FIG. 2 is an example system for performing eye-tracking.

FIG. 3 is a diagram of an example workflow for performing eye-tracking in two stages.

FIG. 4 is a diagram of an example workflow for conditionally performing a first stage of eye-tracking.

FIG. 5 is a diagram of an example workflow for conditionally subsampling a region of interest based on detected movement.

FIG. 6 is a diagram of an example workflow for conditionally subsampling a region of interest based on both detected gaze prediction quality and detected movement.

FIG. 7 is an illustration of example augmented-reality glasses that may be used in connection with embodiments of this disclosure.

FIG. 8 is an illustration of an example virtual-reality headset that may be used in connection with embodiments of this disclosure.

FIG. 9 is an illustration of an example system that incorporates an eye-tracking subsystem capable of tracking a user's eye(s).

FIG. 10 is a more detailed illustration of various aspects of the eye-tracking subsystem illustrated in FIG. 9.

### DETAILED DESCRIPTION OF EXEMPLARY EMBODIMENTS

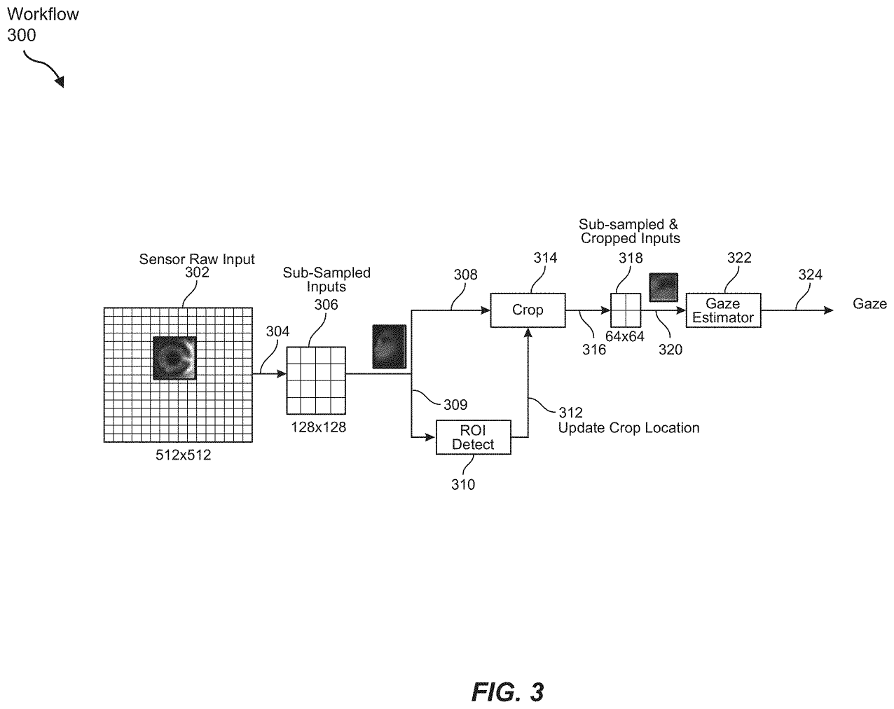

The eye-tracking systems in modern augmented/virtual reality headsets can consume a substantial amount of power. For example, some end-to-end machine learning solutions for eye-tracking consume a few hundredths of milliwatts of power. Three major sources of the power consumption include sensor capture, transmission of captured pixels, and processing the captured pixels through the corresponding machine learning pipeline. Unfortunately, there is significant redundancy in this process as a large portion of the captured and processed pixels do not contribute to eye-tracking. Moreover, a change in the location of the eye region with respect to the camera may break the machine learning component of the eye-tracking pipeline. To address these issues, a two-stage pipeline, as shown in FIG. 3, can be used where in the first stage, a region of interest will be identified and then in a second stage only the region of interest will be used for final gaze prediction. Another additional method for reducing complexity can involve using lower resolution input by subsampling or down-sampling the input pixels. The subsampling or down-sampling procedure may be performed either during or after the sensor captures the pixels. However, the region of interest detection stage itself can consume orders of magnitude higher power and incur significant latency.

The present disclosure is generally directed to improvements to eye-tracking systems in the context of augmented/virtual reality headsets, and in particular to eye-tracking systems that rely upon machine learning to predict a direction of a user's gaze. The disclosed technology may improve upon related systems by reducing power consumption dramatically. For example, the disclosed technology may reduce power consumption by the eye-tracking system from hundreds of milliwatts of power to single-digit milliwatts. The disclosed technology may also reduce latency and improve accuracy in terms of predicting a gaze orientation or direction.

Generally speaking, the disclosed technology may achieve the above-described benefits by reducing a frequency for performing a first stage of an eye-tracking system processing pipeline. This first stage may identify a region of interest. Thus, the region of interest may be detected at a frequency significantly lower than the frequency of a second stage that makes the final prediction of gaze orientation. Nevertheless, the lower frequency cannot always be used without deterioration of eye-tracking performance due to certain corner cases, such as movement caused by users running or jumping, etc. Thus, this application discloses that the lower frequency may be conditionally applied such that, if a corner case is detected, then the first stage may be conditionally activated for an extra frame to improve the accuracy of detecting the region of interest. Thus, the lower frequency may constitute a default or regular frequency that is subject to an exception when a corner case is detected. Similarly, subsampling of just the region of interest, as distinct from subsampling the entire captured frame, may constitute a default or regular procedure that is subject to an exception when a corner case is detected. For example, if either movement is detected or low-quality gaze prediction is detected, then the first stage may be conditionally activated and/or the entire frame may be subsampled, as discussed in more detail below.

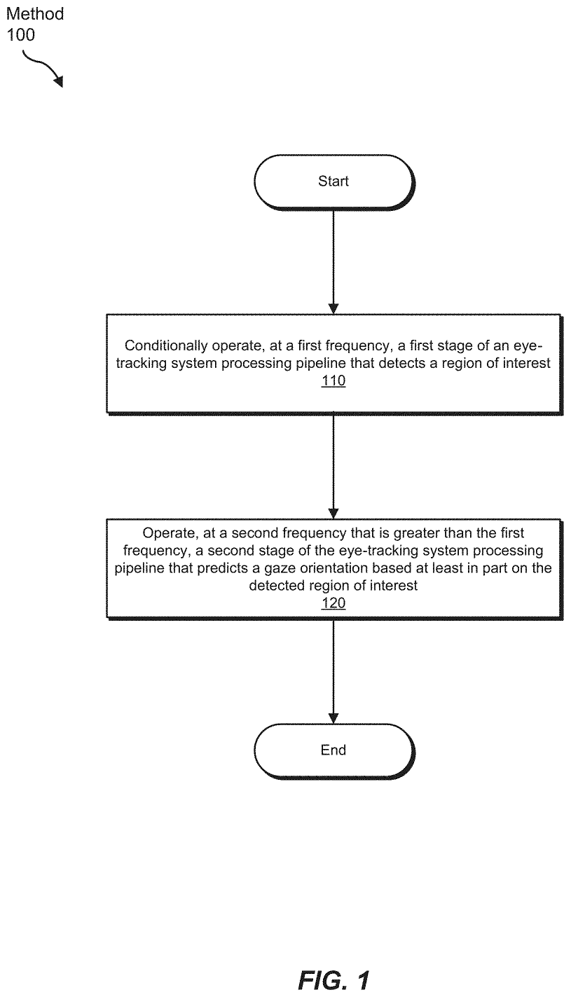

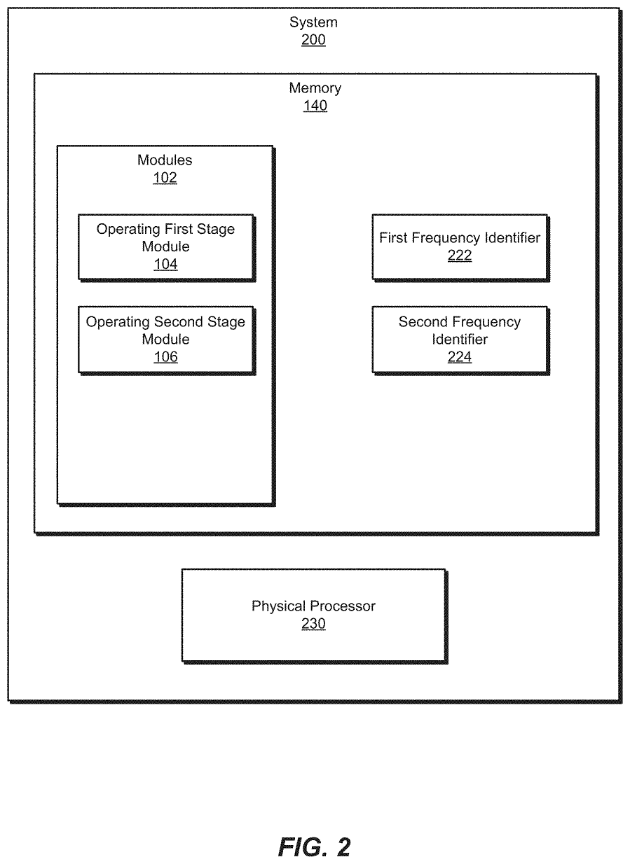

The following will provide, with reference to FIGS. 1-6, detailed descriptions of systems and methods for performing eye-tracking. FIG. 1 is a flow diagram of an exemplary computer-implemented method 100 for performing eye-tracking. The steps shown in FIG. 1 may be performed by any suitable computer-executable code and/or computing system, including a system 200 illustrated in FIG. 2 (which may further include a first frequency identifier 222, a second frequency identifier 224, a physical processor 230, and a memory 140). In one example, each of the steps shown in FIG. 1 may represent an algorithm whose structure includes and/or is represented by multiple sub-steps, examples of which will be provided in greater detail below.

As illustrated in FIG. 1, at step 110 one or more of the systems described herein may conditionally operate, at a first frequency, a first stage of an eye-tracking system processing pipeline that detects a region of interest. For example, at step 110, an operating first stage module 104 (as part of modules 102) may conditionally operate, at a first frequency, a first stage of an eye-tracking system processing pipeline that detects a region of interest.

Operating first stage module 104 may perform step 110 in a variety of ways. Generally speaking, operating first stage module 104 may perform step 110 by setting a frequency for the first stage of the eye-tracking system processing pipeline, during which the region of interest is detected, to be lower than the second frequency of the second stage, during which the gaze orientation is actually predicted. For example, the first frequency may be substantially lower than the second frequency, or may be an order of magnitude lower than the second frequency, for example. In some examples, the first frequency is selected statically based on heuristics gathered from data analysis.

Thus, over a given period of time, such as a minute, the gaze orientation may be predicted more frequently than the region of interest is detected. Thus, if the gaze orientation prediction is being activated again before the region of interest has been updated, then the second stage may simply reuse the previously detected region of interest. Accordingly, the technology of this application may rely upon the design assumption whereby the region of interest remains roughly the same during normal usage of the headset, and the region of interest may only significantly alter or deviate during corner cases, such as cases where the user is moving significantly such as by jumping or running.

The phrase “conditionally operate” may refer to the fact that the first frequency is not universally or exclusively applied but, instead, is applied as a default frequency. In other words, the first frequency is not performed blindly or automatically, but only after checking, at some interval (e.g., the second frequency), whether the first frequency should be deviated from, as discussed at length in connection with FIGS. 3-6 below. Accordingly, operating first stage module 104 may intelligently deviate from the default frequency based upon detection of an indication of a corner case that threatens to impede performance of the eye-tracking system processing pipeline. Similarly, the term “corner case” may refer to cases or scenarios where a higher frequency of performing the first stage becomes desirable or the previously detected region of interest may become less reliable and more likely to have changed, such as in scenarios where the user is running or jumping. Detecting an indication of a corner case may include detecting movement and/or detecting a quality measurement of output of the second stage falling below a threshold.

In some examples detecting the indication of the corner case may be performed by a sensor other than a camera sensor. An illustrative example of such a sensor may include an inertial measurement unit. Additionally, or alternatively, any other suitable sensor, such as an accelerometer or gyroscope may be used, etc.

FIG. 3 shows a workflow 300 for an example two-stage eye-tracking system processing pipeline. As shown in this figure, the region of interest may be universally or always detected (i.e., the first stage) at essentially the same frequency as a gaze estimator makes a prediction for a gaze direction (i.e., the second stage). In particular, a camera sensor may first detect sensor raw input 302 (e.g., 512×512 pixels), and then at step 304 perform a subsampling procedure to generate sub-sampled inputs 306. Subsampling may reduce complexity by eliminating redundancy in cases where the sensor raw input 302 is needlessly or overly detailed for the purposes of eye-tracking.

After sub-sampled inputs 306 are generated, the sub-sampled inputs 306 may proceed along workflow 300, simultaneously or sequentially, at step 308 at which point sub-sampled inputs 306 are cropped using a cropping procedure 314, and at a step 309, at which point a region of interest detection procedure 310 may be performed to detect a corresponding region of interest. Notably, workflow 300 corresponds to a system that generally performs region of interest detection procedure 310 at the same frequency as the subsequent gaze estimation procedure that is discussed further below. The results of performing region of interest detection procedure 310 may be used, at step 312, to update a crop location (e.g., the predicted location of the eyeball within the full frame of sensor raw input 302 and/or the predicted region of interest) that is used to perform cropping procedure 314. The results of performing cropping procedure 314 may, at step 316, result in sub-sampled and cropped inputs 318 (e.g., 64×64 pixels). Subsequently, at step 320, sub-sampled and cropped inputs 318 may be forwarded to a gaze estimator 322, which may estimate an orientation or other description of a corresponding gaze of an eyeball (e.g., the eyeball depicted within sensor raw input 302 in FIG. 3), which may be produced as a result at a step 324. This may correspond to the final output of workflow 300 with respect to eye-tracking.

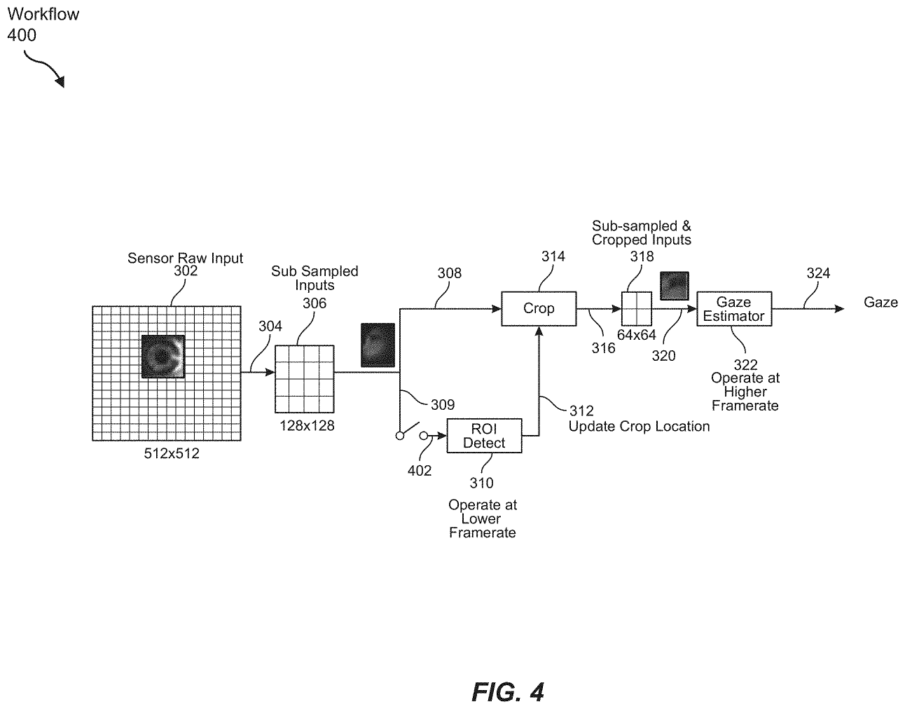

In contrast, FIG. 4 shows a workflow 400 for an updated version of such a two-stage eye-tracking system processing pipeline. In this version, a switch 402 has been inserted prior to region of interest detection procedure 310. This switch may be selectively toggled on or off to break the corresponding path to the region of interest detection procedure, thereby essentially operating the region of interest detection component at a lower frame rate or lower frequency than the gaze estimator component. As discussed above, usage of the switch or lower frequency may rely on the design assumption that the region of interest tends to remain the same or similar during normal usage of the headset. In further examples, such a sensor may actually output a quantitative measurement of movement. Accordingly, detecting movement may include detecting that such a quantitative measurement of movement satisfies a threshold amount of movement, such that sufficiently low or subtle amounts of movement do not necessarily trigger the deviation in terms of frequency of operating the region of interest detection procedure.

Generally speaking, in response to detecting a corner case such as movement, operating first stage module 104 may deviate from the first frequency by activating the first stage (e.g., focused on region of interest detection procedure 310) for an extra frame such that an accuracy of detecting the region of interest is improved. Additionally, or alternatively, in response to detecting the corner case, operating first stage module 104 may also optionally deviate from a subsampling procedure that subsamples the region of interest (e.g., sub-sampling a picture that only predictably contains the eyeball rather than the full frame of camera sensor input) to a subsampling procedure that subsamples an entire frame that includes the region of interest (e.g., sub-sampling the full frame without any cropping to focus on the eyeball itself).

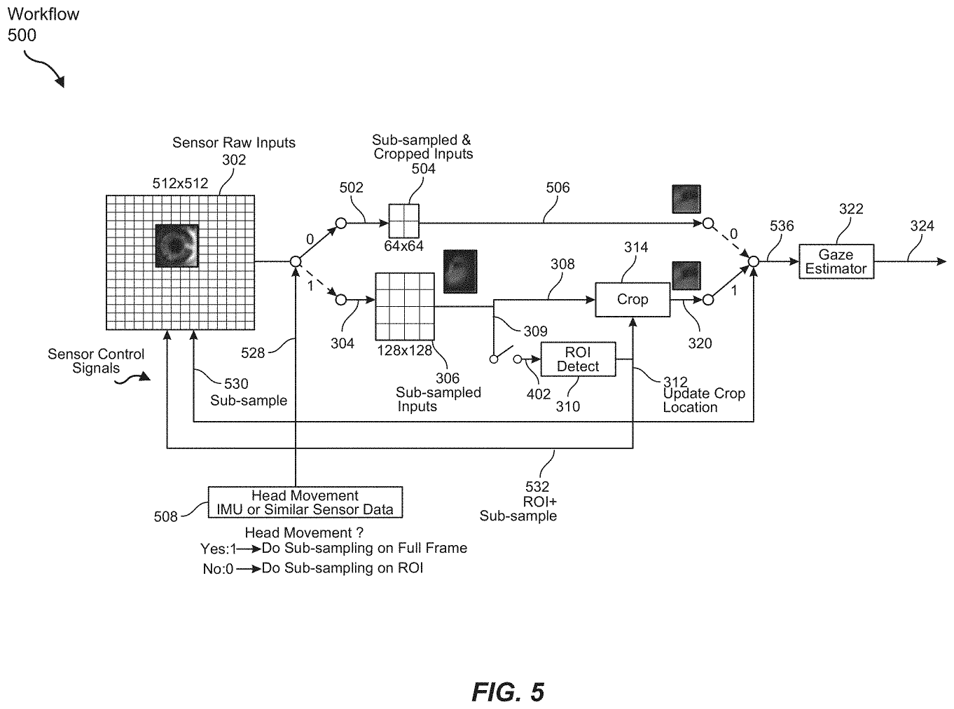

FIG. 5 shows a workflow 500 for deviating to the subsampling procedure that subsamples an entire frame that includes the region of interest. As further shown in this figure, if head movement has been detected by an inertial measurement unit or other similar sensor data (e.g., non-camera sensor data) at a step 508, then subsampling may be performed on the full frame according to updated versions of step 304 and sub-sampled inputs 306 (e.g., 128×128 pixels, corresponding to a sub-sampled, but not cropped, version of sensor raw inputs 302). In contrast, if such movement has not been detected at step 508, then subsampling may be performed on the region of interest itself rather than the full frame according to a step 502, which may generate sub-sampled and cropped inputs 504, which are forwarded at a step 506 and subsequent step 536 to gaze estimator 322 when this path of the workflow is selected at step 508. Consistent with the above, FIG. 5 also shows a step 530 whereby a binary indication from step 508 may be forwarded to a step corresponding to sensor raw inputs 302, thereby determining whether sensor raw inputs 302 should be sub-sampled (e.g., in a case where head movement is not detected) or instead sensor raw inputs 302 should not be sub-sampled (e.g., in a case where head movement is detected). The binary indication from step 508 may also be forwarded, at step 528, to determine which of the two workflow paths (corresponding to step 502 and step 304) should be followed. And the binary indication from step 508 may also be forwarded, at a step 536, to gaze estimator 322, as further shown in FIG. 5. Similarly, at a step 532 a result of region of interest detection procedure 310 may indicate an updated location of the region of interest, and information indicating the updated location may be forwarded to a step corresponding to sensor raw inputs 302, thereby helping to increase accuracy when later extracting the region of interest from sensor raw inputs 302 and/or when performing a sub-sampling operation on corresponding data.

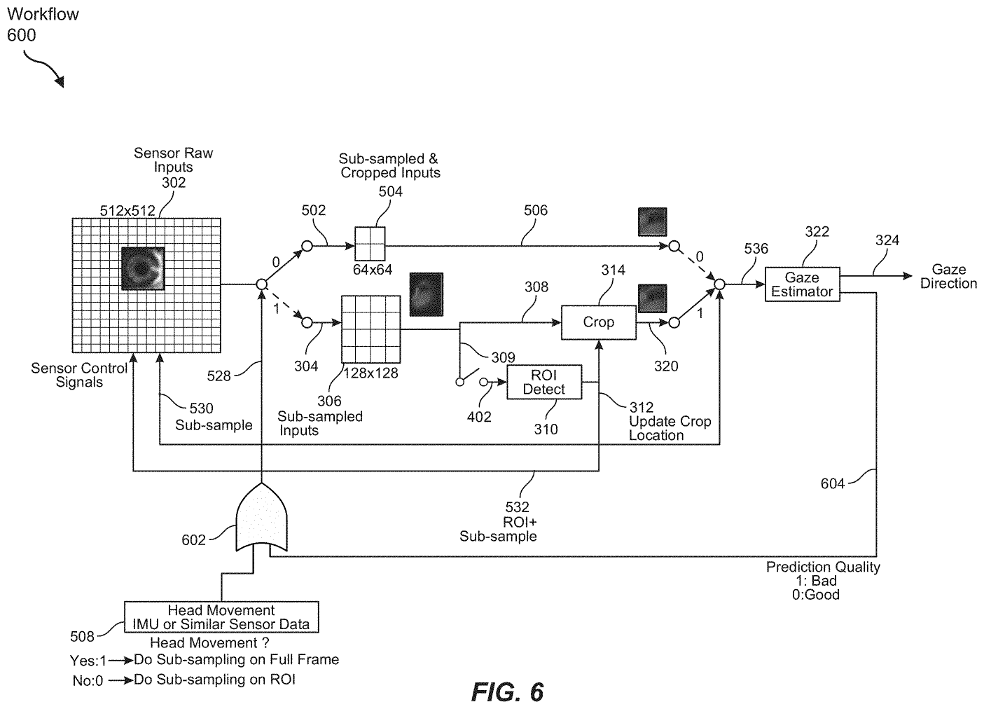

In further examples, the indication of the corner case includes the quality measurement of output of the second stage falling below a threshold. FIG. 6 shows a workflow 600 to help to further illustrate this embodiment. As further shown in this figure, at the conclusion of the second stage of the eye-tracking system processing pipeline, a gaze direction may have been predicted and quality of this prediction may be measured. For example, a numerical measurement of prediction quality may have been compared, at a step 604, against a threshold to arrive at a binary conclusion of either a good prediction or bad prediction. If the prediction quality is determined to be bad, then this may constitute another indication of a corner case such that the subsampling may be performed on the full frame rather than subsampling being performed on the region of interest. Furthermore, in the example of this figure, an OR operation 602 may be executed that, in response to detecting either movement or detecting the quality measurement of output of the second stage falling below the threshold, triggers deviating from a subsampling procedure that subsamples the region of interest to a subsampling procedure that subsamples an entire frame that includes the region of interest.

Returning to FIG. 1, at step 120 one or more of the systems described herein may operate, at a second frequency that is greater than the first frequency, a second stage of the eye-tracking system processing pipeline that predicts a gaze orientation based at least in part on the detected region of interest. For example, at step 120, operating second stage module 106 may operate, at a second frequency that is greater than the first frequency, a second stage of the eye-tracking system processing pipeline that predicts a gaze orientation based at least in part on the detected region of interest.

Operating second stage module 106 may perform step 120 in a variety of ways. Generally speaking, operating second stage module 106 may perform step 120 by simply operating the gaze estimator (see FIG. 4) at a higher frame rate than the region of interest detection component. Unlike the first frequency for the region of interest detection component, the second frequency for the gaze estimator may in some examples remain essentially unconditional or the same over time.

### EXAMPLE EMBODIMENTS

Example 1: An eye-tracking headset apparatus may include a physical processor and at least one physical memory storing executable instructions that, when executed by the physical processor, cause the physical processor to (i) conditionally operate, at a first frequency, a first stage of an eye-tracking system processing pipeline that detects a region of interest and (ii) operate, at a second frequency that is substantially greater than the first frequency, a second stage of the eye-tracking system processing pipeline that predicts a gaze orientation based at least in part on the detected region of interest.

Example 2: The eye-tracking headset apparatus of Example 1, wherein the executable instructions further cause the physical processor to detect an indication of a corner case that threatens to impede performance of the eye-tracking system processing pipeline.

Example 3: The eye-tracking headset apparatus of any of Examples 1-2, wherein the indication of the corner case includes movement or a quality measurement of output of the second stage falling below a threshold.

Example 4: The eye-tracking headset apparatus of any of Examples 1-3 where the indication of the corner case comprises movement.

Example 5: The eye-tracking headset apparatus of any of Examples 1-4 further including an inertial measurement unit that is configured to detect movement.

Example 6: The eye-tracking headset apparatus of any of Examples 1-5 where the inertial measurement unit is configured to detect a quantity of movement that satisfies a predetermined threshold.

Example 7: The eye-tracking headset apparatus of any of Examples 1-6 where the executable instructions further cause the physical processor to deviate, in response to detecting movement, from the first frequency by activating the first stage for an extra frame such that an accuracy of detecting the region of interest is improved.

Example 8: The eye-tracking headset apparatus of any of Examples 1-7 the executable instructions further cause the physical processor to deviate, in response to detecting movement, from a subsampling procedure that subsamples the region of interest to a subsampling procedure that subsamples an entire frame that includes the region of interest.

Example 9: The eye-tracking headset apparatus of any of Examples 1-8 where the indication of the corner case comprises the quality measurement of output of the second stage falling below the threshold.

Example 10: The eye-tracking headset apparatus of any of Examples 1-9 where the executable instructions further cause the physical processor to deviate, in response to detecting the quality measurement of output of the second stage falling below the threshold, from a subsampling procedure that subsamples the region of interest to a subsampling procedure that subsamples an entire frame that includes the region of interest.

Example 11: The eye-tracking headset apparatus of any of Examples 1-10 where the executable instructions further cause the physical processor to execute an inclusive OR operation that, in response to detecting either movement or detecting the quality measurement of output of the second stage falling below the threshold, triggers deviating from a subsampling procedure that subsamples the region of interest to a subsampling procedure that subsamples an entire frame that includes the region of interest.

Example 12: The eye-tracking headset apparatus of any of Examples 1-11 where wherein the executable instructions further cause the physical processor to detect the indication of the corner case that is detected by a sensor other than a camera sensor.

Example 13: The eye-tracking headset apparatus of any of Examples 1-12 where the eye-tracking system processing pipeline is configured to operate as part of a virtual/augmented reality headset.

Example 14: The eye-tracking headset apparatus of any of Examples 1-13 where the executable instructions further cause the physical processor to conditionally operate the first stage of the eye-tracking system processing pipeline that detects the region of interest at the first frequency such that power consumption is achieved that is substantially lower than in comparison to unconditionally operating the first stage.

Example 15: The eye-tracking headset apparatus of any of Examples 1-14 where the executable instructions further cause the physical processor to conditionally operate the first stage of the eye-tracking system processing pipeline that detects the region of interest at the first frequency such that power consumption is reduced from hundreds of milliwatts to single digit milliwatts in comparison to operating the first stage at the second frequency.

Example 16: The eye-tracking headset apparatus of any of Examples 1-15 where the executable instructions further cause the physical processor to operate the first stage of the eye-tracking system processing pipeline that detects the region of interest at the first frequency such that latency is improved in comparison to operating the first stage at the second frequency.

Example 17: The eye-tracking headset apparatus of any of Examples 1-16 where the executable instructions further cause the physical processor to select statically the first frequency based on heuristics gathered from data analysis.

Example 18: The eye-tracking headset apparatus of any of Examples 1-17 where the executable instructions further cause the physical processor to execute a machine learning algorithm to predict the gaze orientation.

Example 19: A computer-implemented method may include conditionally operating, at a first frequency, a first stage of an eye-tracking system processing pipeline that detects a region of interest and operating, at a second frequency that is substantially greater than the first frequency, a second stage of the eye-tracking system processing pipeline that predicts a gaze orientation based at least in part on the detected region of interest.

Example 20: A non-transitory computer-readable medium may include one or more computer-executable instructions that, when executed by at least one processor of a computing device, cause the computing device to conditionally operate, at a first frequency, a first stage of an eye-tracking system processing pipeline that detects a region of interest and operate, at a second frequency that is substantially greater than the first frequency, a second stage of the eye-tracking system processing pipeline that predicts a gaze orientation based at least in part on the detected region of interest.

Embodiments of the present disclosure may include or be implemented in conjunction with various types of artificial-reality systems. Artificial reality is a form of reality that has been adjusted in some manner before presentation to a user, which may include, for example, a virtual reality, an augmented reality, a mixed reality, a hybrid reality, or some combination and/or derivative thereof. Artificial-reality content may include completely computer-generated content or computer-generated content combined with captured (e.g., real-world) content. The artificial-reality content may include video, audio, haptic feedback, or some combination thereof, any of which may be presented in a single channel or in multiple channels (such as stereo video that produces a three-dimensional (3D) effect to the viewer). Additionally, in some embodiments, artificial reality may also be associated with applications, products, accessories, services, or some combination thereof, that are used to, for example, create content in an artificial reality and/or are otherwise used in (e.g., to perform activities in) an artificial reality.

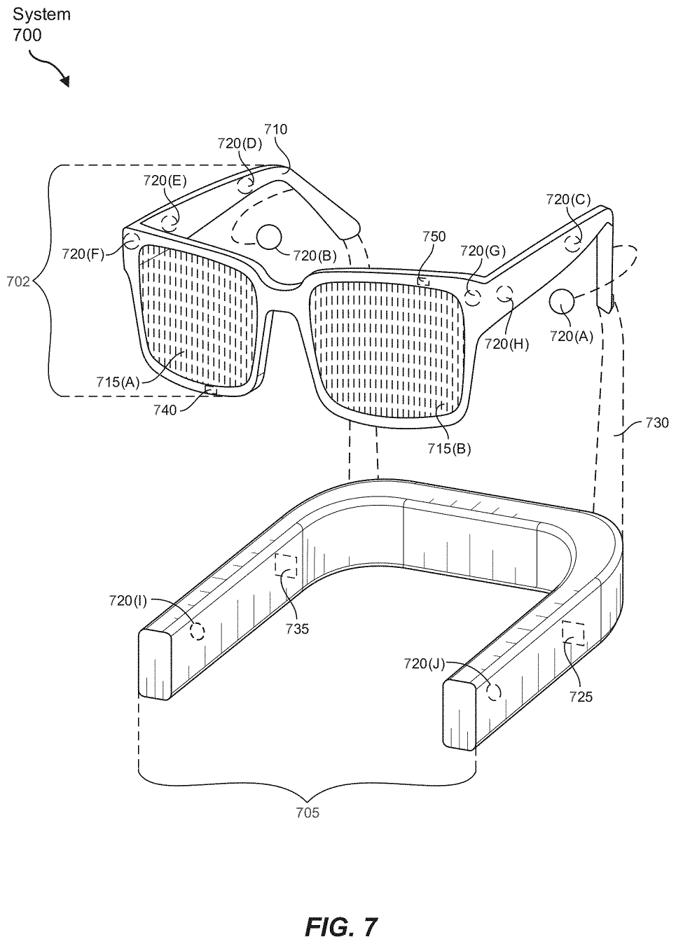

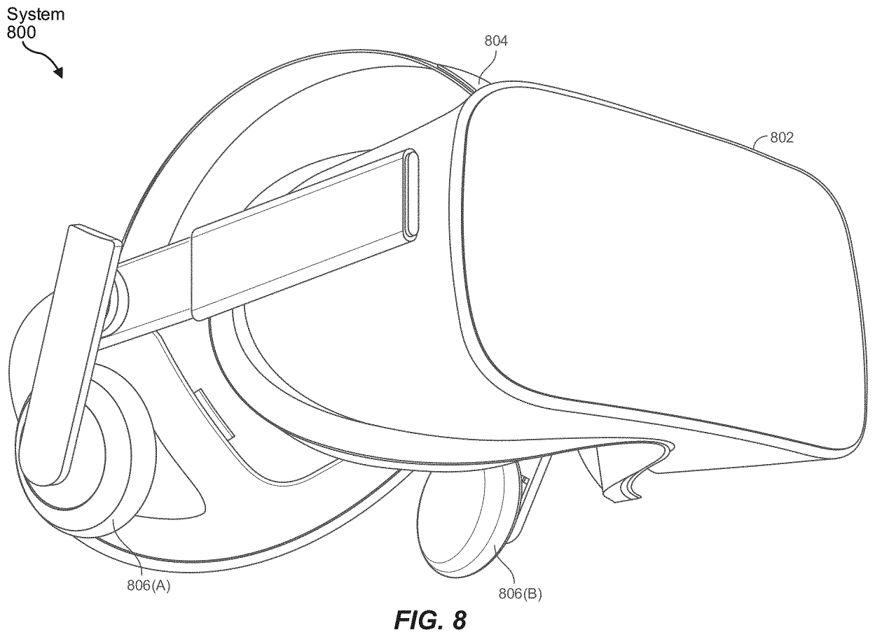

Artificial-reality systems may be implemented in a variety of different form factors and configurations. Some artificial-reality systems may be designed to work without near-eye displays (NEDs). Other artificial-reality systems may include an NED that also provides visibility into the real world (such as, e.g., augmented-reality system 700 in FIG. 7) or that visually immerses a user in an artificial reality (such as, e.g., virtual-reality system 800 in FIG. 8). While some artificial-reality devices may be self-contained systems, other artificial-reality devices may communicate and/or coordinate with external devices to provide an artificial-reality experience to a user. Examples of such external devices include handheld controllers, mobile devices, desktop computers, devices worn by a user, devices worn by one or more other users, and/or any other suitable external system.

Turning to FIG. 7, augmented-reality system 700 may include an eyewear device 702 with a frame 710 configured to hold a left display device 715(A) and a right display device 715(B) in front of a user's eyes. Display devices 715(A) and 715(B) may act together or independently to present an image or series of images to a user. While augmented-reality system 700 includes two displays, embodiments of this disclosure may be implemented in augmented-reality systems with a single NED or more than two NEDs.

In some embodiments, augmented-reality system 700 may include one or more sensors, such as sensor 740. Sensor 740 may generate measurement signals in response to motion of augmented-reality system 700 and may be located on substantially any portion of frame 710. Sensor 740 may represent one or more of a variety of different sensing mechanisms, such as a position sensor, an inertial measurement unit (IMU), a depth camera assembly, a structured light emitter and/or detector, or any combination thereof. In some embodiments, augmented-reality system 700 may or may not include sensor 740 or may include more than one sensor. In embodiments in which sensor 740 includes an IMU, the IMU may generate calibration data based on measurement signals from sensor 740. Examples of sensor 740 may include, without limitation, accelerometers, gyroscopes, magnetometers, other suitable types of sensors that detect motion, sensors used for error correction of the IMU, or some combination thereof.

In some examples, augmented-reality system 700 may also include a microphone array with a plurality of acoustic transducers 720(A)-720(J), referred to collectively as acoustic transducers 720. Acoustic transducers 720 may represent transducers that detect air pressure variations induced by sound waves. Each acoustic transducer 720 may be configured to detect sound and convert the detected sound into an electronic format (e.g., an analog or digital format). The microphone array in FIG. 7 may include, for example, ten acoustic transducers: 720(A) and 720(B), which may be designed to be placed inside a corresponding ear of the user, acoustic transducers 720(C), 720(D), 720(E), 720(F), 720(G), and 720(H), which may be positioned at various locations on frame 710, and/or acoustic transducers 720(I) and 720(J), which may be positioned on a corresponding neckband 705.

In some embodiments, one or more of acoustic transducers 720(A)-(J) may be used as output transducers (e.g., speakers). For example, acoustic transducers 720(A) and/or 720(B) may be earbuds or any other suitable type of headphone or speaker.

The configuration of acoustic transducers 720 of the microphone array may vary. While augmented-reality system 700 is shown in FIG. 7 as having ten acoustic transducers 720, the number of acoustic transducers 720 may be greater or less than ten. In some embodiments, using higher numbers of acoustic transducers 720 may increase the amount of audio information collected and/or the sensitivity and accuracy of the audio information. In contrast, using a lower number of acoustic transducers 720 may decrease the computing power required by an associated controller 750 to process the collected audio information. In addition, the position of each acoustic transducer 720 of the microphone array may vary. For example, the position of an acoustic transducer 720 may include a defined position on the user, a defined coordinate on frame 710, an orientation associated with each acoustic transducer 720, or some combination thereof.

Acoustic transducers 720(A) and 720(B) may be positioned on different parts of the user's ear, such as behind the pinna, behind the tragus, and/or within the auricle or fossa. Or, there may be additional acoustic transducers 720 on or surrounding the ear in addition to acoustic transducers 720 inside the ear canal. Having an acoustic transducer 720 positioned next to an ear canal of a user may enable the microphone array to collect information on how sounds arrive at the ear canal. By positioning at least two of acoustic transducers 720 on either side of a user's head (e.g., as binaural microphones), augmented-reality device 700 may simulate binaural hearing and capture a 3D stereo sound field around about a user's head. In some embodiments, acoustic transducers 720(A) and 720(B) may be connected to augmented-reality system 700 via a wired connection 730, and in other embodiments acoustic transducers 720(A) and 720(B) may be connected to augmented-reality system 700 via a wireless connection (e.g., a BLUETOOTH connection). In still other embodiments, acoustic transducers 720(A) and 720(B) may not be used at all in conjunction with augmented-reality system 700.

Acoustic transducers 720 on frame 710 may be positioned in a variety of different ways, including along the length of the temples, across the bridge, above or below display devices 715(A) and 715(B), or some combination thereof. Acoustic transducers 720 may also be oriented such that the microphone array is able to detect sounds in a wide range of directions surrounding the user wearing the augmented-reality system 700. In some embodiments, an optimization process may be performed during manufacturing of augmented-reality system 700 to determine relative positioning of each acoustic transducer 720 in the microphone array.

In some examples, augmented-reality system 700 may include or be connected to an external device (e.g., a paired device), such as neckband 705. Neckband 705 generally represents any type or form of paired device. Thus, the following discussion of neckband 705 may also apply to various other paired devices, such as charging cases, smart watches, smart phones, wrist bands, other wearable devices, hand-held controllers, tablet computers, laptop computers, other external compute devices, etc.

As shown, neckband 705 may be coupled to eyewear device 702 via one or more connectors. The connectors may be wired or wireless and may include electrical and/or non-electrical (e.g., structural) components. In some cases, eyewear device 702 and neckband 705 may operate independently without any wired or wireless connection between them. While FIG. 7 illustrates the components of eyewear device 702 and neckband 705 in example locations on eyewear device 702 and neckband 705, the components may be located elsewhere and/or distributed differently on eyewear device 702 and/or neckband 705. In some embodiments, the components of eyewear device 702 and neckband 705 may be located on one or more additional peripheral devices paired with eyewear device 702, neckband 705, or some combination thereof.

Pairing external devices, such as neckband 705, with augmented-reality eyewear devices may enable the eyewear devices to achieve the form factor of a pair of glasses while still providing sufficient battery and computation power for expanded capabilities. Some or all of the battery power, computational resources, and/or additional features of augmented-reality system 700 may be provided by a paired device or shared between a paired device and an eyewear device, thus reducing the weight, heat profile, and form factor of the eyewear device overall while still retaining desired functionality. For example, neckband 705 may allow components that would otherwise be included on an eyewear device to be included in neckband 705 since users may tolerate a heavier weight load on their shoulders than they would tolerate on their heads. Neckband 705 may also have a larger surface area over which to diffuse and disperse heat to the ambient environment. Thus, neckband 705 may allow for greater battery and computation capacity than might otherwise have been possible on a standalone eyewear device. Since weight carried in neckband 705 may be less invasive to a user than weight carried in eyewear device 702, a user may tolerate wearing a lighter eyewear device and carrying or wearing the paired device for greater lengths of time than a user would tolerate wearing a heavy standalone eyewear device, thereby enabling users to more fully incorporate artificial-reality environments into their day-to-day activities.

Neckband 705 may be communicatively coupled with eyewear device 702 and/or to other devices. These other devices may provide certain functions (e.g., tracking, localizing, depth mapping, processing, storage, etc.) to augmented-reality system 700. In the embodiment of FIG. 7, neckband 705 may include two acoustic transducers (e.g., 720(I) and 720(J)) that are part of the microphone array (or potentially form their own microphone subarray). Neckband 705 may also include a controller 725 and a power source 735.

Acoustic transducers 720(I) and 720(J) of neckband 705 may be configured to detect sound and convert the detected sound into an electronic format (analog or digital). In the embodiment of FIG. 7, acoustic transducers 720(I) and 720(J) may be positioned on neckband 705, thereby increasing the distance between the neckband acoustic transducers 720(I) and 720(J) and other acoustic transducers 720 positioned on eyewear device 702. In some cases, increasing the distance between acoustic transducers 720 of the microphone array may improve the accuracy of beamforming performed via the microphone array. For example, if a sound is detected by acoustic transducers 720(C) and 720(D) and the distance between acoustic transducers 720(C) and 720(D) is greater than, e.g., the distance between acoustic transducers 720(D) and 720(E), the determined source location of the detected sound may be more accurate than if the sound had been detected by acoustic transducers 720(D) and 720(E).

Controller 725 of neckband 705 may process information generated by the sensors on neckband 705 and/or augmented-reality system 700. For example, controller 725 may process information from the microphone array that describes sounds detected by the microphone array. For each detected sound, controller 725 may perform a direction-of-arrival (DOA) estimation to estimate a direction from which the detected sound arrived at the microphone array. As the microphone array detects sounds, controller 725 may populate an audio data set with the information. In embodiments in which augmented-reality system 700 includes an inertial measurement unit, controller 725 may compute all inertial and spatial calculations from the IMU located on eyewear device 702. A connector may convey information between augmented-reality system 700 and neckband 705 and between augmented-reality system 700 and controller 725. The information may be in the form of optical data, electrical data, wireless data, or any other transmittable data form. Moving the processing of information generated by augmented-reality system 700 to neckband 705 may reduce weight and heat in eyewear device 702, making it more comfortable to the user.

Power source 735 in neckband 705 may provide power to eyewear device 702 and/or to neckband 705. Power source 735 may include, without limitation, lithium ion batteries, lithium-polymer batteries, primary lithium batteries, alkaline batteries, or any other form of power storage. In some cases, power source 735 may be a wired power source. Including power source 735 on neckband 705 instead of on eyewear device 702 may help better distribute the weight and heat generated by power source 735.

As noted, some artificial-reality systems may, instead of blending an artificial reality with actual reality, substantially replace one or more of a user's sensory perceptions of the real world with a virtual experience. One example of this type of system is a head-worn display system, such as virtual-reality system 800 in FIG. 8, that mostly or completely covers a user's field of view. Virtual-reality system 800 may include a front rigid body 802 and a band 804 shaped to fit around a user's head. Virtual-reality system 800 may also include output audio transducers 806(A) and 806(B). Furthermore, while not shown in FIG. 8, front rigid body 802 may include one or more electronic elements, including one or more electronic displays, one or more inertial measurement units (IMUs), one or more tracking emitters or detectors, and/or any other suitable device or system for creating an artificial-reality experience.

Artificial-reality systems may include a variety of types of visual feedback mechanisms. For example, display devices in augmented-reality system 700 and/or virtual-reality system 800 may include one or more liquid crystal displays (LCDs), light emitting diode (LED) displays, microLED displays, organic LED (OLED) displays, digital light project (DLP) micro-displays, liquid crystal on silicon (LCoS) micro-displays, and/or any other suitable type of display screen. These artificial-reality systems may include a single display screen for both eyes or may provide a display screen for each eye, which may allow for additional flexibility for varifocal adjustments or for correcting a user's refractive error. Some of these artificial-reality systems may also include optical subsystems having one or more lenses (e.g., conventional concave or convex lenses, Fresnel lenses, adjustable liquid lenses, etc.) through which a user may view a display screen. These optical subsystems may serve a variety of purposes, including to collimate (e.g., make an object appear at a greater distance than its physical distance), to magnify (e.g., make an object appear larger than its actual size), and/or to relay (to, e.g., the viewer's eyes) light. These optical subsystems may be used in a non-pupil-forming architecture (such as a single lens configuration that directly collimates light but results in so-called pincushion distortion) and/or a pupil-forming architecture (such as a multi-lens configuration that produces so-called barrel distortion to nullify pincushion distortion).

In addition to or instead of using display screens, some of the artificial-reality systems described herein may include one or more projection systems. For example, display devices in augmented-reality system 700 and/or virtual-reality system 800 may include microLED projectors that project light (using, e.g., a waveguide) into display devices, such as clear combiner lenses that allow ambient light to pass through. The display devices may refract the projected light toward a user's pupil and may enable a user to simultaneously view both artificial-reality content and the real world. The display devices may accomplish this using any of a variety of different optical components, including waveguide components (e.g., holographic, planar, diffractive, polarized, and/or reflective waveguide elements), light-manipulation surfaces and elements (such as diffractive, reflective, and refractive elements and gratings), coupling elements, etc. Artificial-reality systems may also be configured with any other suitable type or form of image projection system, such as retinal projectors used in virtual retina displays.

The artificial-reality systems described herein may also include various types of computer vision components and subsystems. For example, augmented-reality system 700 and/or virtual-reality system 800 may include one or more optical sensors, such as two-dimensional (2D) or 3D cameras, structured light transmitters and detectors, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions.

The artificial-reality systems described herein may also include one or more input and/or output audio transducers. Output audio transducers may include voice coil speakers, ribbon speakers, electrostatic speakers, piezoelectric speakers, bone conduction transducers, cartilage conduction transducers, tragus-vibration transducers, and/or any other suitable type or form of audio transducer. Similarly, input audio transducers may include condenser microphones, dynamic microphones, ribbon microphones, and/or any other type or form of input transducer. In some embodiments, a single transducer may be used for both audio input and audio output.

In some embodiments, the artificial-reality systems described herein may also include tactile (i.e., haptic) feedback systems, which may be incorporated into headwear, gloves, body suits, handheld controllers, environmental devices (e.g., chairs, floormats, etc.), and/or any other type of device or system. Haptic feedback systems may provide various types of cutaneous feedback, including vibration, force, traction, texture, and/or temperature. Haptic feedback systems may also provide various types of kinesthetic feedback, such as motion and compliance. Haptic feedback may be implemented using motors, piezoelectric actuators, fluidic systems, and/or a variety of other types of feedback mechanisms. Haptic feedback systems may be implemented independent of other artificial-reality devices, within other artificial-reality devices, and/or in conjunction with other artificial-reality devices.

By providing haptic sensations, audible content, and/or visual content, artificial-reality systems may create an entire virtual experience or enhance a user's real-world experience in a variety of contexts and environments. For instance, artificial-reality systems may assist or extend a user's perception, memory, or cognition within a particular environment. Some systems may enhance a user's interactions with other people in the real world or may enable more immersive interactions with other people in a virtual world. Artificial-reality systems may also be used for educational purposes (e.g., for teaching or training in schools, hospitals, government organizations, military organizations, business enterprises, etc.), entertainment purposes (e.g., for playing video games, listening to music, watching video content, etc.), and/or for accessibility purposes (e.g., as hearing aids, visual aids, etc.). The embodiments disclosed herein may enable or enhance a user's artificial-reality experience in one or more of these contexts and environments and/or in other contexts and environments.

In some embodiments, the systems described herein may also include an eye-tracking subsystem designed to identify and track various characteristics of a user's eye(s), such as the user's gaze direction. The phrase “eye tracking” may, in some examples, refer to a process by which the position, orientation, and/or motion of an eye is measured, detected, sensed, determined, and/or monitored. The disclosed systems may measure the position, orientation, and/or motion of an eye in a variety of different ways, including through the use of various optical-based eye-tracking techniques, ultrasound-based eye-tracking techniques, etc. An eye-tracking subsystem may be configured in a number of different ways and may include a variety of different eye-tracking hardware components or other computer-vision components. For example, an eye-tracking subsystem may include a variety of different optical sensors, such as two-dimensional (2D) or 3D cameras, time-of-flight depth sensors, single-beam or sweeping laser rangefinders, 3D LiDAR sensors, and/or any other suitable type or form of optical sensor. In this example, a processing subsystem may process data from one or more of these sensors to measure, detect, determine, and/or otherwise monitor the position, orientation, and/or motion of the user's eye(s).

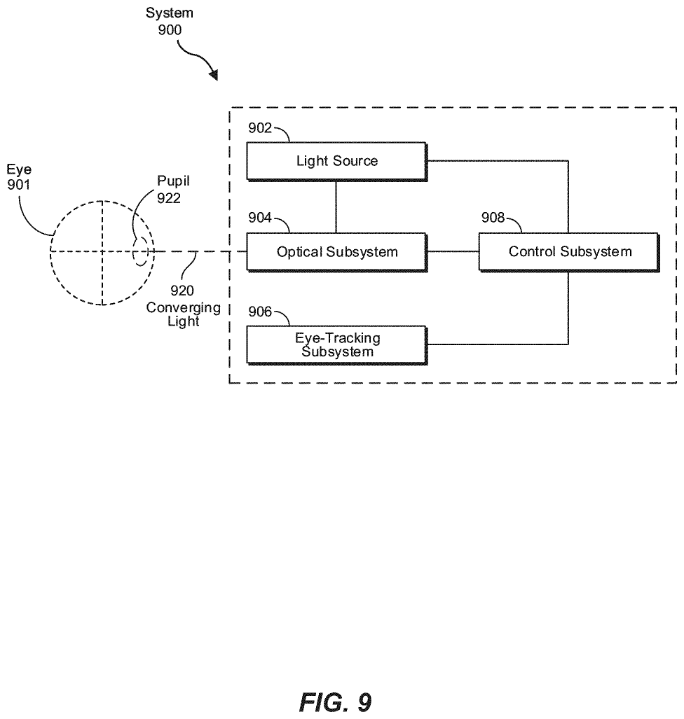

FIG. 9 is an illustration of an exemplary system 900 that incorporates an eye-tracking subsystem capable of tracking a user's eye(s). As depicted in FIG. 9, system 900 may include a light source 902, an optical subsystem 904, an eye-tracking subsystem 906, and/or a control subsystem 908. In some examples, light source 902 may generate light for an image (e.g., to be presented to an eye 901 of the viewer). Light source 902 may represent any of a variety of suitable devices. For example, light source 902 can include a two-dimensional projector (e.g., a LCoS display), a scanning source (e.g., a scanning laser), or other device (e.g., an LCD, an LED display, an OLED display, an active-matrix OLED display (AMOLED), a transparent OLED display (TOLED), a waveguide, or some other display capable of generating light for presenting an image to the viewer). In some examples, the image may represent a virtual image, which may refer to an optical image formed from the apparent divergence of light rays from a point in space, as opposed to an image formed from the light ray's actual divergence.

In some embodiments, optical subsystem 904 may receive the light generated by light source 902 and generate, based on the received light, converging light 920 that includes the image. In some examples, optical subsystem 904 may include any number of lenses (e.g., Fresnel lenses, convex lenses, concave lenses), apertures, filters, mirrors, prisms, and/or other optical components, possibly in combination with actuators and/or other devices. In particular, the actuators and/or other devices may translate and/or rotate one or more of the optical components to alter one or more aspects of converging light 920. Further, various mechanical couplings may serve to maintain the relative spacing and/or the orientation of the optical components in any suitable combination.

In one embodiment, eye-tracking subsystem 906 may generate tracking information indicating a gaze angle of an eye 901 of the viewer. In this embodiment, control subsystem 908 may control aspects of optical subsystem 904 (e.g., the angle of incidence of converging light 920) based at least in part on this tracking information. Additionally, in some examples, control subsystem 908 may store and utilize historical tracking information (e.g., a history of the tracking information over a given duration, such as the previous second or fraction thereof) to anticipate the gaze angle of eye 901 (e.g., an angle between the visual axis and the anatomical axis of eye 901). In some embodiments, eye-tracking subsystem 906 may detect radiation emanating from some portion of eye 901 (e.g., the cornea, the iris, the pupil, or the like) to determine the current gaze angle of eye 901. In other examples, eye-tracking subsystem 906 may employ a wavefront sensor to track the current location of the pupil.

Any number of techniques can be used to track eye 901. Some techniques may involve illuminating eye 901 with infrared light and measuring reflections with at least one optical sensor that is tuned to be sensitive to the infrared light. Information about how the infrared light is reflected from eye 901 may be analyzed to determine the position(s), orientation(s), and/or motion(s) of one or more eye feature(s), such as the cornea, pupil, iris, and/or retinal blood vessels.

In some examples, the radiation captured by a sensor of eye-tracking subsystem 906 may be digitized (i.e., converted to an electronic signal). Further, the sensor may transmit a digital representation of this electronic signal to one or more processors (for example, processors associated with a device including eye-tracking subsystem 906). Eye-tracking subsystem 906 may include any of a variety of sensors in a variety of different configurations. For example, eye-tracking subsystem 906 may include an infrared detector that reacts to infrared radiation. The infrared detector may be a thermal detector, a photonic detector, and/or any other suitable type of detector. Thermal detectors may include detectors that react to thermal effects of the incident infrared radiation.

In some examples, one or more processors may process the digital representation generated by the sensor(s) of eye-tracking subsystem 906 to track the movement of eye 901. In another example, these processors may track the movements of eye 901 by executing algorithms represented by computer-executable instructions stored on non-transitory memory. In some examples, on-chip logic (e.g., an application-specific integrated circuit or ASIC) may be used to perform at least portions of such algorithms. As noted, eye-tracking subsystem 906 may be programmed to use an output of the sensor(s) to track movement of eye 901. In some embodiments, eye-tracking subsystem 906 may analyze the digital representation generated by the sensors to extract eye rotation information from changes in reflections. In one embodiment, eye-tracking subsystem 906 may use corneal reflections or glints (also known as Purkinje images) and/or the center of the eye's pupil 922 as features to track over time.

In some embodiments, eye-tracking subsystem 906 may use the center of the eye's pupil 922 and infrared or near-infrared, non-collimated light to create corneal reflections. In these embodiments, eye-tracking subsystem 906 may use the vector between the center of the eye's pupil 922 and the corneal reflections to compute the gaze direction of eye 901. In some embodiments, the disclosed systems may perform a calibration procedure for an individual (using, e.g., supervised or unsupervised techniques) before tracking the user's eyes. For example, the calibration procedure may include directing users to look at one or more points displayed on a display while the eye-tracking system records the values that correspond to each gaze position associated with each point.

In some embodiments, eye-tracking subsystem 906 may use two types of infrared and/or near-infrared (also known as active light) eye-tracking techniques: bright-pupil and dark-pupil eye tracking, which may be differentiated based on the location of an illumination source with respect to the optical elements used. If the illumination is coaxial with the optical path, then eye 901 may act as a retroreflector as the light reflects off the retina, thereby creating a bright pupil effect similar to a red-eye effect in photography. If the illumination source is offset from the optical path, then the eye's pupil 922 may appear dark because the retroreflection from the retina is directed away from the sensor. In some embodiments, bright-pupil tracking may create greater iris/pupil contrast, allowing more robust eye tracking with iris pigmentation, and may feature reduced interference (e.g., interference caused by eyelashes and other obscuring features). Bright-pupil tracking may also allow tracking in lighting conditions ranging from total darkness to a very bright environment.

In some embodiments, control subsystem 908 may control light source 902 and/or optical subsystem 904 to reduce optical aberrations (e.g., chromatic aberrations and/or monochromatic aberrations) of the image that may be caused by or influenced by eye 901. In some examples, as mentioned above, control subsystem 908 may use the tracking information from eye-tracking subsystem 906 to perform such control. For example, in controlling light source 902, control subsystem 908 may alter the light generated by light source 902 (e.g., by way of image rendering) to modify (e.g., pre-distort) the image so that the aberration of the image caused by eye 901 is reduced.

The disclosed systems may track both the position and relative size of the pupil (since, e.g., the pupil dilates and/or contracts). In some examples, the eye-tracking devices and components (e.g., sensors and/or sources) used for detecting and/or tracking the pupil may be different (or calibrated differently) for different types of eyes. For example, the frequency range of the sensors may be different (or separately calibrated) for eyes of different colors and/or different pupil types, sizes, and/or the like. As such, the various eye-tracking components (e.g., infrared sources and/or sensors) described herein may need to be calibrated for each individual user and/or eye.

The disclosed systems may track both eyes with and without ophthalmic correction, such as that provided by contact lenses worn by the user. In some embodiments, ophthalmic correction elements (e.g., adjustable lenses) may be directly incorporated into the artificial reality systems described herein. In some examples, the color of the user's eye may necessitate modification of a corresponding eye-tracking algorithm. For example, eye-tracking algorithms may need to be modified based at least in part on the differing color contrast between a brown eye and, for example, a blue eye.

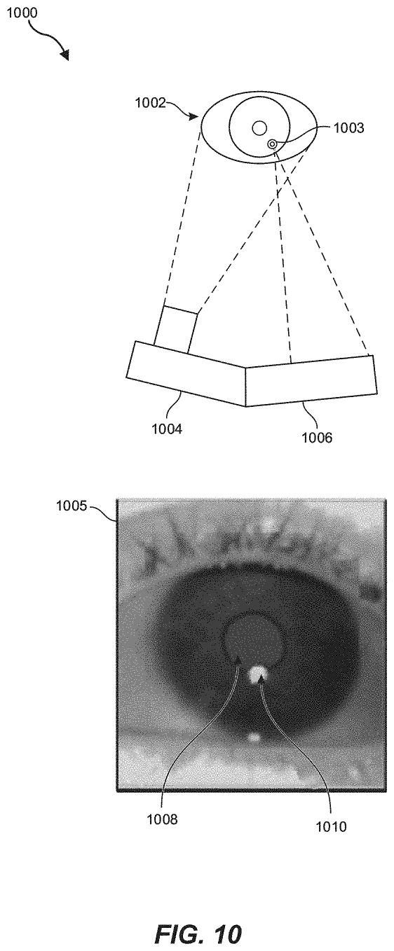

FIG. 10 is a more detailed illustration of various aspects of the eye-tracking subsystem illustrated in FIG. 9. As shown in this figure, an eye-tracking subsystem 1000 may include at least one source 1004 and at least one sensor 1006. Source 1004 generally represents any type or form of element capable of emitting radiation. In one example, source 1004 may generate visible, infrared, and/or near-infrared radiation. In some examples, source 1004 may radiate non-collimated infrared and/or near-infrared portions of the electromagnetic spectrum towards an eye 1002 of a user. Source 1004 may utilize a variety of sampling rates and speeds. For example, the disclosed systems may use sources with higher sampling rates in order to capture fixational eye movements of a user's eye 1002 and/or to correctly measure saccade dynamics of the user's eye 1002. As noted above, any type or form of eye-tracking technique may be used to track the user's eye 1002, including optical-based eye-tracking techniques, ultrasound-based eye-tracking techniques, etc.

Sensor 1006 generally represents any type or form of element capable of detecting radiation, such as radiation reflected off the user's eye 1002. Examples of sensor 1006 include, without limitation, a charge coupled device (CCD), a photodiode array, a complementary metal-oxide-semiconductor (CMOS) based sensor device, and/or the like. In one example, sensor 1006 may represent a sensor having predetermined parameters, including, but not limited to, a dynamic resolution range, linearity, and/or other characteristic selected and/or designed specifically for eye tracking.

As detailed above, eye-tracking subsystem 1000 may generate one or more glints. As detailed above, a glint 1003 may represent reflections of radiation (e.g., infrared radiation from an infrared source, such as source 1004) from the structure of the user's eye. In various embodiments, glint 1003 and/or the user's pupil may be tracked using an eye-tracking algorithm executed by a processor (either within or external to an artificial reality device). For example, an artificial reality device may include a processor and/or a memory device in order to perform eye tracking locally and/or a transceiver to send and receive the data necessary to perform eye tracking on an external device (e.g., a mobile phone, cloud server, or other computing device).

FIG. 10 shows an example image 1005 captured by an eye-tracking subsystem, such as eye-tracking subsystem 1000. In this example, image 1005 may include both the user's pupil 1008 and a glint 1010 near the same. In some examples, pupil 1008 and/or glint 1010 may be identified using an artificial-intelligence-based algorithm, such as a computer-vision-based algorithm. In one embodiment, image 1005 may represent a single frame in a series of frames that may be analyzed continuously in order to track the eye 1002 of the user. Further, pupil 1008 and/or glint 1010 may be tracked over a period of time to determine a user's gaze.

In one example, eye-tracking subsystem 1000 may be configured to identify and measure the inter-pupillary distance (IPD) of a user. In some embodiments, eye-tracking subsystem 1000 may measure and/or calculate the IPD of the user while the user is wearing the artificial reality system. In these embodiments, eye-tracking subsystem 1000 may detect the positions of a user's eyes and may use this information to calculate the user's IPD.

As noted, the eye-tracking systems or subsystems disclosed herein may track a user's eye position and/or eye movement in a variety of ways. In one example, one or more light sources and/or optical sensors may capture an image of the user's eyes. The eye-tracking subsystem may then use the captured information to determine the user's inter-pupillary distance, interocular distance, and/or a 3D position of each eye (e.g., for distortion adjustment purposes), including a magnitude of torsion and rotation (i.e., roll, pitch, and yaw) and/or gaze directions for each eye. In one example, infrared light may be emitted by the eye-tracking subsystem and reflected from each eye. The reflected light may be received or detected by an optical sensor and analyzed to extract eye rotation data from changes in the infrared light reflected by each eye.

The eye-tracking subsystem may use any of a variety of different methods to track the eyes of a user. For example, a light source (e.g., infrared light-emitting diodes) may emit a dot pattern onto each eye of the user. The eye-tracking subsystem may then detect (e.g., via an optical sensor coupled to the artificial reality system) and analyze a reflection of the dot pattern from each eye of the user to identify a location of each pupil of the user. Accordingly, the eye-tracking subsystem may track up to six degrees of freedom of each eye (i.e., 3D position, roll, pitch, and yaw) and at least a subset of the tracked quantities may be combined from two eyes of a user to estimate a gaze point (i.e., a 3D location or position in a virtual scene where the user is looking) and/or an IPD.

In some cases, the distance between a user's pupil and a display may change as the user's eye moves to look in different directions. The varying distance between a pupil and a display as viewing direction changes may be referred to as “pupil swim” and may contribute to distortion perceived by the user as a result of light focusing in different locations as the distance between the pupil and the display changes. Accordingly, measuring distortion at different eye positions and pupil distances relative to displays and generating distortion corrections for different positions and distances may allow mitigation of distortion caused by pupil swim by tracking the 3D position of a user's eyes and applying a distortion correction corresponding to the 3D position of each of the user's eyes at a given point in time. Thus, knowing the 3D position of each of a user's eyes may allow for the mitigation of distortion caused by changes in the distance between the pupil of the eye and the display by applying a distortion correction for each 3D eye position. Furthermore, as noted above, knowing the position of each of the user's eyes may also enable the eye-tracking subsystem to make automated adjustments for a user's IPD.

In some embodiments, a display subsystem may include a variety of additional subsystems that may work in conjunction with the eye-tracking subsystems described herein. For example, a display subsystem may include a varifocal subsystem, a scene-rendering module, and/or a vergence-processing module. The varifocal subsystem may cause left and right display elements to vary the focal distance of the display device. In one embodiment, the varifocal subsystem may physically change the distance between a display and the optics through which it is viewed by moving the display, the optics, or both. Additionally, moving or translating two lenses relative to each other may also be used to change the focal distance of the display. Thus, the varifocal subsystem may include actuators or motors that move displays and/or optics to change the distance between them. This varifocal subsystem may be separate from or integrated into the display subsystem. The varifocal subsystem may also be integrated into or separate from its actuation subsystem and/or the eye-tracking subsystems described herein.

In one example, the display subsystem may include a vergence-processing module configured to determine a vergence depth of a user's gaze based on a gaze point and/or an estimated intersection of the gaze lines determined by the eye-tracking subsystem. Vergence may refer to the simultaneous movement or rotation of both eyes in opposite directions to maintain single binocular vision, which may be naturally and automatically performed by the human eye. Thus, a location where a user's eyes are verged is where the user is looking and is also typically the location where the user's eyes are focused. For example, the vergence-processing module may triangulate gaze lines to estimate a distance or depth from the user associated with intersection of the gaze lines. The depth associated with intersection of the gaze lines may then be used as an approximation for the accommodation distance, which may identify a distance from the user where the user's eyes are directed. Thus, the vergence distance may allow for the determination of a location where the user's eyes should be focused and a depth from the user's eyes at which the eyes are focused, thereby providing information (such as an object or plane of focus) for rendering adjustments to the virtual scene.

The vergence-processing module may coordinate with the eye-tracking subsystems described herein to make adjustments to the display subsystem to account for a user's vergence depth. When the user is focused on something at a distance, the user's pupils may be slightly farther apart than when the user is focused on something close. The eye-tracking subsystem may obtain information about the user's vergence or focus depth and may adjust the display subsystem to be closer together when the user's eyes focus or verge on something close and to be farther apart when the user's eyes focus or verge on something at a distance.

The eye-tracking information generated by the above-described eye-tracking subsystems may also be used, for example, to modify various aspect of how different computer-generated images are presented. For example, a display subsystem may be configured to modify, based on information generated by an eye-tracking subsystem, at least one aspect of how the computer-generated images are presented. For instance, the computer-generated images may be modified based on the user's eye movement, such that if a user is looking up, the computer-generated images may be moved upward on the screen. Similarly, if the user is looking to the side or down, the computer-generated images may be moved to the side or downward on the screen. If the user's eyes are closed, the computer-generated images may be paused or removed from the display and resumed once the user's eyes are back open.

The above-described eye-tracking subsystems can be incorporated into one or more of the various artificial reality systems described herein in a variety of ways. For example, one or more of the various components of system 900 and/or eye-tracking subsystem 1000 may be incorporated into augmented-reality system 700 in FIG. 7 and/or virtual-reality system 800 in FIG. 8 to enable these systems to perform various eye-tracking tasks (including one or more of the eye-tracking operations described herein).

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

As detailed above, the computing devices and systems described and/or illustrated herein broadly represent any type or form of computing device or system capable of executing computer-readable instructions, such as those contained within the modules described herein. In their most basic configuration, these computing device(s) may each include at least one memory device and at least one physical processor.

In some examples, the term “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the modules described and/or illustrated herein may represent portions of a single module or application. In addition, in certain embodiments one or more of these modules may represent one or more software applications or programs that, when executed by a computing device, may cause the computing device to perform one or more tasks. For example, one or more of the modules described and/or illustrated herein may represent modules stored and configured to run on one or more of the computing devices or systems described and/or illustrated herein. One or more of these modules may also represent all or portions of one or more special-purpose computers configured to perform one or more tasks.

In addition, one or more of the modules described herein may transform data, physical devices, and/or representations of physical devices from one form to another. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

In some embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to any claims appended hereto and their equivalents in determining the scope of the present disclosure.

Unless otherwise noted, the terms “connected to” and “coupled to” (and their derivatives), as used in the specification and/or claims, are to be construed as permitting both direct and indirect (i.e., via other elements or components) connection. In addition, the terms “a” or “an,” as used in the specification and/or claims, are to be construed as meaning “at least one of.” Finally, for ease of use, the terms “including” and “having” (and their derivatives), as used in the specification and/or claims, are interchangeable with and have the same meaning as the word “comprising.”

What is claimed is:

1. An eye-tracking headset comprising:
a physical processor; and
at least one physical memory storing executable instructions that, when executed by the physical processor, cause the physical processor to:
conditionally operate, at a first frequency, a first stage of an eye-tracking system processing pipeline that detects a region of interest; and
operate, at a second frequency that is substantially greater than the first frequency, a second stage of the eye-tracking system processing pipeline that predicts a gaze orientation based at least in part on the detected region of interest; wherein:
the first stage updates information identifying a crop location corresponding to a human eye; and
the second stage performs, at the second frequency that is substantially greater than the first frequency, a cropping procedure using the crop location previously updated from the first stage that is operating at the first frequency.

2. The eye-tracking headset of claim 1, wherein the executable instructions further cause the physical processor to detect an indication of a corner case that threatens to impede performance of the eye-tracking system processing pipeline.

3. The eye-tracking headset of claim 2, wherein the indication of the corner case comprises:
movement; or
a quality measurement of output of the second stage falling below a threshold.

4. The eye-tracking headset of claim 3, wherein the indication of the corner case comprises movement.

5. The eye-tracking headset of claim 4, further comprising an inertial measurement unit that is configured to detect movement.

6. The eye-tracking headset of claim 5, wherein the inertial measurement unit is configured to detect a quantity of movement that satisfies a predetermined threshold.

7. The eye-tracking headset of claim 4, wherein the executable instructions further cause the physical processor to deviate, in response to detecting movement, from the first frequency by activating the first stage for an extra frame such that an accuracy of detecting the region of interest is improved.

8. The eye-tracking headset of claim 4, wherein the executable instructions further cause the physical processor to deviate, in response to detecting movement, from a subsampling procedure that subsamples the region of interest to a subsampling procedure that subsamples an entire frame that includes the region of interest.

9. The eye-tracking headset of claim 3, wherein the indication of the corner case comprises the quality measurement of output of the second stage falling below the threshold.

10. The eye-tracking headset of claim 9, wherein the executable instructions further cause the physical processor to deviate, in response to detecting the quality measurement of output of the second stage falling below the threshold, from a subsampling procedure that subsamples the region of interest to a subsampling procedure that subsamples an entire frame that includes the region of interest.

11. The eye-tracking headset of claim 3, wherein the executable instructions further cause the physical processor to execute an inclusive OR operation that, in response to detecting either movement or detecting the quality measurement of output of the second stage falling below the threshold, triggers deviating from a subsampling procedure that subsamples the region of interest to a subsampling procedure that subsamples an entire frame that includes the region of interest.

12. The eye-tracking headset of claim 3, wherein the executable instructions further cause the physical processor to detect the indication of the corner case that is detected by a sensor other than a camera sensor.

13. The eye-tracking headset of claim 1, wherein the eye-tracking system processing pipeline is configured to operate as part of a virtual/augmented reality headset.

14. The eye-tracking headset of claim 1, wherein the executable instructions further cause the physical processor to conditionally operate the first stage of the eye-tracking system processing pipeline that detects the region of interest at the first frequency such that power consumption is achieved that is substantially lower than in comparison to unconditionally operating the first stage.

15. The eye-tracking headset of claim 14, wherein the executable instructions further cause the physical processor to conditionally operate the first stage of the eye-tracking system processing pipeline that detects the region of interest at the first frequency such that power consumption is reduced from hundreds of milliwatts to single digit milliwatts in comparison to operating the first stage at the second frequency.

16. The eye-tracking headset of claim 1, wherein the executable instructions further cause the physical processor to operate the first stage of the eye-tracking system processing pipeline that detects the region of interest at the first frequency such that latency is improved in comparison to operating the first stage at the second frequency.

17. The eye-tracking headset of claim 1, wherein the executable instructions further cause the physical processor to select statically the first frequency based on heuristics gathered from data analysis.

18. The eye-tracking headset of claim 1, wherein the executable instructions further cause the physical processor to execute a machine learning algorithm to predict the gaze orientation.

19. A method comprising:
conditionally operating, at a first frequency, a first stage of an eye-tracking system processing pipeline that detects a region of interest; and
operating, at a second frequency that is substantially greater than the first frequency, a second stage of the eye-tracking system processing pipeline that predicts a gaze orientation based at least in part on the detected region of interest;
wherein:
the first stage updates information identifying a crop location corresponding to a human eye; and
the second stage performs, at the second frequency that is substantially greater than the first frequency, a cropping procedure using the crop location previously updated from the first stage that is operating at the first frequency.

20. A non-transitory computer-readable medium comprising one or more computer-executable instructions that, when executed by at least one processor of a computing device, cause the computing device to:
conditionally operate, at a first frequency, a first stage of an eye-tracking system processing pipeline that detects a region of interest; and
operate, at a second frequency that is substantially greater than the first frequency, a second stage of the eye-tracking system processing pipeline that predicts a gaze orientation based at least in part on the detected region of interest;
wherein:
the first stage updates information identifying a crop location corresponding to a human eye; and
the second stage performs, at the second frequency that is substantially greater than the first frequency, a cropping procedure using the crop location previously updated from the first stage that is operating at the first frequency.
